(19) United States
(12) Patent Application Publication
(10) Pub. No.: US 2021/0323559 A1
(43) Pub. Date: Oct. 21, 2021

(54) DATA PROCESSING DEVICE, HUMAN-MACHINE INTERFACE SYSTEM INCLUDING THE DEVICE, VEHICLE INCLUDING THE SYSTEM, METHOD FOR EVALUATING USER DISCOMFORT, AND COMPUTER-READABLE MEDIUM FOR CARRYING OUT THE METHOD

(71) Applicants: TOYOTA JIDOSHA KABUSHIKI KAISHA, Toyota-shi (JP); UNIVERSITÉ GUSTAVE EIFFEL, Mame-Ia-Vallée Cedex 2 (FR)

(72) Inventors: Marleen DE WESER, Wilsele (BE); Christophe JALLAIS, Lyon (FR); Catherine BERTHELON, Lyon (FR); Alexandra FORT, Lyon (FR); Antonio HIDALGO, Toulouse (FR); Adolphe BEQUET, Bron (FR); Fabien MOREAU, Bron (FR); Hélène TATTEGRAIN, Lyon (FR); Morgane EVIN, Marseille (FR)

(21) Appl. No.: 17/231,128

(22) Filed: Apr. 15, 2021

(30) Foreign Application Priority Data
Apr. 17, 2020 (EP) 20315192.3

(51) Int. Cl. B60W 40/09; G10L 25/63; G06K 9/00; G08B 21/18; G06N 20/00; G06N 5/04

(52) U.S. Cl. CPC B60W 40/09; G10L 25/63; G06K 9/00335; G06K 9/00845; B60W 2040/0872; G08B 21/182; G06N 20/00; G06N 5/04; G06K 9/00604

(57) ABSTRACT

The present disclosure concerns a data processing device configured to be connected to one or more physiological sensors for receiving physiological data of a user, to one or more behavioral sensors for receiving behavioral data of the user and to a vehicle context detection unit for receiving vehicle context information, and to determine a discomfort level index value based on a personality profile of the user together with at least the vehicle context information and physiological and behavioral data received within corresponding time windows, as well as a method for evaluating user discomfort implemented by said data processing device.

### CROSS-REFERENCE TO RELATED APPLICATION

This application claims priority to European Patent Application No. 20315192.3 filed on Apr. 17, 2020, incorporated herein by reference in its entirety.

### BACKGROUND

### 1. Technical Field

The disclosure relates to a data processing device, a human-machine interface system including the device, a vehicle including the system, a method for evaluating user discomfort, and a computer-readable medium for carrying out the method.

### 2. Description of Related Art

The automotive industry is increasingly introducing Advanced Driver Assistance Systems (ADAS) designed to improve driving safety by reducing the risk of human error. These embedded systems can interact with the driver in different ways, either by giving the driver additional information on the state of the environment (via a multi-modal communication interface: audio, visual, tactile, etc.), or by processing information about the driver's mental state such as stress, fatigue, vigilance or drowsiness in order to assist him and/or to prevent potential risks (cf. Jinjun Wang et al, 2010: “Real-time driving danger-level prediction”, Engineering Applications of Artificial Intelligence; Volume 23, Issue 8, December 2010, Pages 1247-1254). These existing systems process individually stress, fatigue, vigilance or drowsiness by considering behavioral or physiological parameters separately.

However, it is desirable to not only measure one specific mental state of the driver (fatigue or drowsiness, etc.) but to measure the feeling or experience of discomfort in driving. Meng and Siren (2012) propose that discomfort in driving can be considered as a form of awareness of the driver of changes in his/her own driving ability (cf. Meng and Siren (2012): “Cognitive problems, self-rated changes in driving skills, driving-related discomfort and self-regulation of driving in old drivers” Accident Analysis & Prevention, Volume 49, November 2012, Pages 322-329). Thus, these authors define the feeling of discomfort as related to a feeling of anxiety as a consequence of being self-aware that one's driving ability in a specific driving situation (for example driving in heavy rain) may include a potential driving risk. This feeling of discomfort may increase in time and can lead to complete avoidance of specific driving situations. However, Meng and Siren only analyze subjective data using questionnaires without considering measuring the (objective) driver behavioral or physiological data. In addition, most of the research dealing with (dis)comfort in driving, addresses this notion mainly in terms of physical ergonomics of the driving cabin (posture, driver seat dimensions, etc.).

It is further known to measure the driver's mental state by electrophysiological and/or driver behavioral measures. For example, WO2008/0127465 A1 discloses a system which predicts driving danger by capturing vehicle dynamic parameters, driver physiological data and driver behavior features; applying a learning algorithm to the features; and predicting driving danger. However, the system only predicts the driving danger of the current driving situation, in order to warn a driver before an undesirable event as e.g. an accident happens. It has also been proposed, for example in KR 20170094836 A, US 2018/0174457, CN 109017797 or CN 107662611 A, to measure the emotional state of the driver, on the basis of physiological data, driving behavior and/or emotional cues, and eventually to change the driving mode accordingly. On the other hand, in WO 2018/014953 A1, it was proposed to use physiological and behavioral data, together with vehicle context data, to determine a discomfort level index value, and output a warning and/or perform driver assistance if this value exceeds a reference index.

However, these previously proposed devices and methods fail to take into consideration endogenous psychological factors which are not necessarily reflected in physiological or behavioral data or even in emotional cues, but will influence the driver's subjective discomfort, with a potentially serious impact on driving safety.

### SUMMARY

A first aspect of the present disclosure relates to a data processing device configured to be connected to one or more physiological sensors for receiving physiological data of a user, to one or more behavioral sensors for receiving behavioral data of the user and to a vehicle context detection unit for receiving vehicle context information, and to determine a discomfort level index value based on a personality profile of the user together with at least the vehicle context information, the physiological data and the behavioral data received within corresponding time windows. The discomfort level index value may be an integer from a scale graded by either ascending or descending level of discomfort. It could thus be alternatively understood as a comfort index level value. The user may be a vehicle driver or passenger, for example in a partially or fully automated vehicle.

Consequently, the endogenous influence of the user's own personality on his subjective discomfort level can be more adequately reflected, together with exogenous factors such as the vehicle context, on the discomfort level index value determined by the data processing device.

The data processing device may be configured to be also connected to an emotional state detection unit for receiving emotional state information concerning the user, and to determine the discomfort level index value based also on the emotional state information concerning the user. In particular, the emotional state detection unit may be configured to detect emotional cues in images and/or sound received from a camera and/or a microphone connected to the emotional state detection unit. Consequently, not only the user's baseline personality profile may be reflected in the discomfort level index value, but also his transient emotional state.

The personality profile may comprise an index value associated to each of one or more personality traits, which may in particular include openness, agreeableness, extraversion, neuroticism, and/or conscientiousness, that is, the “Big Five” personality traits, and/or trait anxiety as measured for instance by the State-Trait Anxiety Inventory (S TAI).

The data processing device may be configured to apply a learning classification algorithm, such as e.g. any one of random forest, J48, naïve Bayesian, deep neural network or recurrent neural network algorithms, for determining the discomfort level index value. Consequently, this algorithm may be first taught in a learning phase to establish correlations between discomfort level and vehicle context information, physiological and behavioral data and personality profile, as well as eventually also emotional state data, in a learning phase using experimental data from one or more user subjects and one or more driving events, so as to subsequently apply those correlations in determining the comfort level index value on the basis of those parameters.

The one or more physiological sensors may include one or more electro-dermal sensors, and the data processing device may be configured to receive, as the physiological data from the one or more electro-dermal sensors, amplitude and/or latency of skin conductivity responses. The data processing device may in particular be configured to determine the discomfort index value on the basis of i.a. the number of occurrences of phasic skin conductivity responses within a corresponding time window, their mean amplitude, the standard deviation of their amplitude, their mean latency and/or the standard deviation of their latency.

Alternatively or complementarily to the electro-dermal sensors, the one or more physiological sensors may include one or more cardiac sensors, and the data processing device may be configured to receive RR intervals as the physiological data from the one or more cardiac sensors. The data processing device may in particular be configured to determine the discomfort index value on the basis of i.a. the minimum RR interval, the maximum RR interval, the mean RR interval, the standard deviation of the RR intervals, and/or the square-rooted standard deviation of the RR intervals, within a corresponding time window.

Alternatively or complementarily to the electro-dermal sensors and/or cardiac sensors, the one or more physiological sensors may include one or more eye tracking sensors, and the data processing device may be configured to receive occurrence and/or duration of eye fixations as the physiological data from the one or more eye tracking sensors. The data processing device may in particular be configured to determine the discomfort index value on the basis of i.a. the number of occurrences of eye fixations within a corresponding time window, their total duration, their mean duration, and/or the standard deviation of their duration.

The data processing device may in particular be also configured to output a warning and/or perform user assistance if the discomfort level index value exceeds a predetermined threshold. This may help ensure the user's comfort following future driving events, by preventing a negative long-term impact on the emotional and cognitive state of the user.

A second aspect of the present disclosure relates to a human-machine interface system including the data processing device of the first aspect, as well as the one or more physiological sensors, the one or more behavioral sensors, and the vehicle context detection unit, each connected to the data processing device.

A third aspect of the present disclosure relates to a vehicle including the human-machine interface system of the second aspect.

A fourth aspect of the present disclosure relates to a computer-implemented method for evaluating user discomfort, including the steps of receiving physiological data of a user from one or more physiological sensors; receiving behavioral data of the user from one or more behavioral sensors; receiving vehicle context information from a vehicle context detection unit; and determining a discomfort level index value based on a personality profile of the user, together with at least the physiological data, the behavioral data and the vehicle context information received within corresponding time windows.

A fifth aspect of the present disclosure relates to computer-readable medium including instructions which, when executed by a computer, cause the computer to carry out the computer-implemented method of the fourth aspect.

The above summary of some aspects is not intended to describe each disclosed embodiment or every implementation of the disclosure. In particular, selected features of any illustrative embodiment within this specification may be incorporated into an additional embodiment unless clearly stated to the contrary.

### BRIEF DESCRIPTION OF THE DRAWINGS

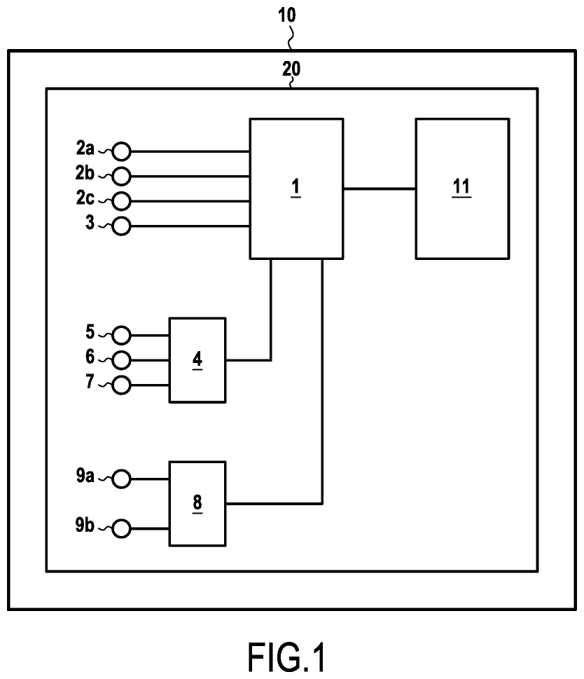

The disclosure may be more completely understood in consideration of the following detailed description of various embodiments in connection with the accompanying FIG. 1, which shows a block diagram of a vehicle according to embodiments of the present disclosure.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION OF EMBODIMENTS

For the following defined terms, these definitions shall be applied, unless a different definition is given in the claims or elsewhere in this specification.

All numeric values are herein assumed to be preceded by the term “about”, whether or not explicitly indicated. The term “about” generally refers to a range of numbers that one of skill in the art would consider equivalent to the recited value (i.e. having the same function or result). In many instances, the term “about” may be indicative as including numbers that are rounded to the nearest significant figure.

Any recitation of numerical ranges by endpoints includes all numbers within that range (e.g., 1 to 5 includes a.o. 1, 4/3, 1.5, 2, e, 2.75, 3, π, 3.80, 4, and 5).

Although some suitable dimension ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

FIG. 1 shows a block diagram of a data processing device 1 according to embodiments of the present disclosure. The data processing device 1 may be part of a human-machine interface (HMI) system 20. The HMI system 20 may be part of a vehicle 10, and in particular provide a driving interface for a human user of the vehicle 10.

The data processing device 1 may be connected to and/or comprise a data storage for storing a reference data set. The data processing device 1 may comprise an electronic circuit, a processor (shared, dedicated, or group), a combinational logic circuit, a memory that executes one or more software programs, and/or other suitable components that provide the described functionality. The data processing device 1 may be configured to additionally carry out further functions in the vehicle 10. For example, the data processing device 1 may also be configured to act as a general purpose electronic control unit (ECU) of the vehicle 10.

The HMI system 20 may also comprise one or several physiological sensors 2a,2b,2c to which the data processing device 1 may be connected to receive a data output thereof. Each physiological sensor 2 may be configured to sense at least one physiological feature of the user. For example, the physiological sensors 2a,2b,2c electro-dermal may include one or more electro-dermal sensors 2a, one or more cardiac sensors 2b, and/or one or more eye tracking sensors 2c. The one or more electro-dermal sensors 2a may comprise Ag/AgCl electrodes, located for example at least at the level of the second phalanx of the index and the third digit of the non-dominant hand of the user and be configured to measure a skin conductance level (SCL). The one or more cardiac sensors 2b may also comprise Ag/AgCl electrodes and be configured to measure an electrocardiographic (ECG) signal. The one or more eye tracking sensors 2c may comprise, for instance, eye tracking Pupilabs® v2 glasses, with a tracking frequency of 120 Hz, a gaze accuracy of 1.0°, a gaze precision of 0.08°, a camera latency of 5.7 ms, and a processing latency of 3-4 ms.

The SCL may be sampled, for instance, at a 1000 Hz sampling rate, and the one or more electro-dermal sensors 2a may be further configured to detect, from the SCL signal, the occurrence of phasic skin conductivity responses and measure their amplitude and/or their latency to transmit these, as physiological data, to the data processing device 1. Alternatively, however, the one or more electro-dermal sensors 2a may directly transmit the SCL signal to the data processing device 1, which may then be itself configured to detect the occurrence of phasic skin conductivity responses and measure their amplitude and/or their latency.

The ECG signal may also be sampled, for instance, at a 1000 Hz sampling rate, and the one or more cardiac sensors 2b may be further configured to detect, from the ECG signal, the individual heartbeats and measure the RR intervals between them, to transmit these, as physiological data, to the data processing device. Alternatively, however, the one or more cardiac sensors 2b may directly transmit the ECG signal to the data processing device 1, which may then be itself configured to detect the individual heartbeats and measure the RR intervals between them.

The eye tracking sensors 2c may be configured to detect occurrence and duration of eye fixations, and transmit them, as physiological data, to the data processing device 1. Alternatively however, the eye tracking sensors 2c may directly transmit pupil position data to the data processing device, which may be configured to detect occurrence and duration of eye fixations from these eye position data.

The HMI system 20 may also comprise one or more behavioral sensors 3. The one or more behavioral sensors 3 may be configured to measure one or more behavioral features of the user, in particular concerning driving behavior, and transmit it to the data processing device 1. For example, the one or more behavioral sensors 3 may measure the vehicle's lateral position on the road (e.g. supported by GPS) and/or steering wheel angle. All behavioral data may be sampled at a 60 Hz sampling rate.

Moreover, the HMI system 20 may also comprise a vehicle context detection unit 4. The vehicle context detection unit 4 may be configured to determine vehicle context information describing a current driving situation and transmit it to the data processing device 1. Such driving situations may include e.g. a situation in which the vehicle 10 comprising the HMI system 20 overtakes another vehicle, a situation in the vehicle 10 comprising the HMI system 20 is approaching a traffic light, a situation in which the vehicle 10 comprising the HMI system 20 approaches a pedestrian or a pedestrian approaches the vehicle 10, a situation in the vehicle 10 comprising the HMI system 20 is driven in the dark, a situation in the vehicle 10 comprising the HMI system 20 makes a left-turn (in right-hand traffic), a situation in which the vehicle 10 comprising the HMI system 20 is in dense traffic and/or facing aggressive and/or unexpected behavior by other road users or other driving situations which may challenge the comfort level of the user, such as, for instance heavy rain, snowfall or icing.

In order to determine the vehicle context information, the vehicle context detection unit 4 may be connected to a plurality of sensors configured to monitor the environment of the vehicle, e.g. a GPS 5, a radar 6 or one or several outside cameras 7. The vehicle context detection unit 4 may also be connected to the one or more behavioral sensors 3 for receiving their output. For example, based on the information received from a GPS, cameras and an electronic map providing a scheduled driving route, the vehicle context detection unit 4 can determine a left turn of the vehicle.

The HMI system 20 may also comprise an emotional state detection unit 8. The emotional state detection unit 8 may be configured to determine emotional state information describing a current emotional state of the user and transmit it to the data processing device 1. Such emotional state information may comprise an emotional state index value associated to each one of one or more emotions, such as, e.g. sadness, interest, anxiety, alertness, boredom, impatience, anger, serenity, fear and/or happiness.

In order to determine the emotional state information, the emotional state detection unit 8 may be connected to one or more sensors configured to monitor the emotional state of the user, e.g. an interior camera 9a and/or a microphone 9b, in order to receive information from them, such as images and/or sound, containing emotional cues. Such emotional cues may be contained e.g. in acoustic features of speech like timbre, prosody and rhythm and/or visual features linked to facial expression. These acoustic and/or visual features may be correlated to specific emotions using machine learning algorithms trained on datasets such as e.g. the Danish Emotional Speech database, the Berlin Database of Emotional Speech, and/or the AffectNet annotated facial expression database, so that the emotional state detection unit 8 may infer the emotional state information from the images and/or sound received from the camera 9a and/or microphone 9b. Alternatively or complementarily to those sensors, the emotional state detection unit 8 may also be connected to the physiological and/or behavioral sensors for receiving their output and using it to infer the emotional state information.

The vehicle context detection unit 4 and/or the emotional state detection unit 8 may each be an electronic control device, as e.g. a processor. Each one of them, or both, may also be provided by the general purpose ECU of the vehicle. It is further possible that the data processing device 1 comprises the vehicle context detection unit 4 and/or the emotional state detection unit 8.

The data processing device 1 may be configured to calculate, based on physiological data received from the one or more electro-dermal sensors 2a over an electro-dermal time window TW_EDA, i.a. the number N_scrs, mean amplitude Amp_moy, standard deviation of the amplitude Et_Amp_Scrs, mean latency Lat_Moy_scrs and/or standard deviation of the latency ET_lat_scrs of the phasic skin conductivity responses within this time window TW_EDA. Furthermore, the data processing device 1 may be configured to calculate, based on physiological data received from the one or more cardiac sensors 2b over a cardiac time window TW_ECG, i.a. the minimum RR interval RRint_min, the maximum RR interval RRint_max, the mean RR interval RRint_moy, the standard deviation SDNN of the RR intervals, and/or the room-mean-squared standard deviation RMSSD of the RR intervals, within this cardiac time window TW_ECG. Additionally or alternatively, the data processing device 1 may be configured to calculate, based on physiological data received from the one or more eye tracking sensors 2c over an eye tracking time window TW_ET, i.a. the number nbFix of occurrences of eye fixations within this eye tracking time window TW_ET, their total duration durationFixTot, their mean duration durationFixMean, and/or the standard deviation durationFixSD of their duration.

Furthermore, the data processing device 1 may be configured to calculate, based on behavioral data received from the one or more behavioral sensors 3 within a behavioral time window TW_BE, i.a. the standard deviation SDLP of the vehicle's lateral position on the road, the standard deviation SDWA of the steering wheel angle, and/or the steering wheel reversal rate SRR.

A personality profile of the user may be stored within the data storage connected to and/or comprised within the data processing device 1. This personality profile may comprise an index value associated to each of one or more personality traits, such as openness, agreeableness, extraversion, neuroticism, conscientiousness, and/or trait anxiety and may have been previously calculated e.g. on the basis of responses of the user to a questionnaire and/or physiological, behavioral, emotional and/or vehicle context data gathered over one or more drives and correlated to one or more of the personality traits by a learning algorithm. This learning algorithm may have been trained and optimized to select significantly correlated features from which the personality traits can be inferred on the basis of a generic dataset with data from multiple users in multiple situations.

The data processing device 1 may be configured to determine a vehicle context event based on the vehicle context information, and then a discomfort index level value for the discomfort resulting from this vehicle context event. Such vehicle context events may be e.g. overtaking another vehicle, approaching a traffic light, approaching a pedestrian, driving in the dark, or making a left-turn (in right-hand traffic). The discomfort level index value may take the form of an integer, e.g. an integer value between 1 and 5, between 1 and 3, or just one of 1 or 2.

More specifically, the data processing device 1 may be configured to apply a learning classification algorithm, such as e.g. a random forest, J48, naïve Bayesian, deep neural network or recurrent neural network algorithm, in order to determine the discomfort level index value based on the personality profile of the user together with the vehicle context information received from the vehicle context detection unit 4, the physiological and behavioral data received, within the corresponding time windows TW_EDA, TW_ECG, TW_ET and TW_BE, from the physiological and behavioral sensors 2a-2c and 3, as well as, eventually, the emotional state information received from the emotional state detection unit 8. The learning classification algorithm may have been trained and/or updated using a generic dataset linked to multiple users and/or one specifically linked to the current user. This learning classification algorithm may have been chosen and optimized based on an index created from a weighting of available indices such as the AUC-ROC curve, precision, recall, TF/FP rate, Matthews correlation coefficient, and/or precision recall curves and subsequently optimized selecting the input features and corresponding time windows best correlated to the discomfort level index value.

The data processing device 1 may be further configured to output a warning and/or trigger user assistance if the discomfort level index value exceeds a predetermined threshold DT. For this purpose, the data processing device may for instance be connected to an advanced user assistance system (ADAS) 11 within the HMI system 20. The ADAS 11 may even be configured to gradually increase driving assistance, i.e. increasingly take over driving tasks, as the discomfort level index value determined by the data processing device increases, i.e. the user's discomfort increases. The ADAS 11 may even be capable of automated driving, e.g. level 2 partial automation, level 3 conditional automation, or even higher.

Three different methods for evaluating user discomfort have been tested in a driving simulator using correspondingly configured HMI systems 20. In each example, a different learning classification algorithm and different sets of physiological and behavioral data and personality traits have been applied. In each case, the learning classification algorithm has been trained on 59 different user test subjects. The three methods were then tested on 10 different user test subjects for validation. During both training and validation, the test subjects were exposed to various driving situations and asked to indicate their resulting discomfort level on a scale of 1 to 5. Their personality profiles were established using questionnaires.

In the first example, the data processing device 1 determined the discomfort level index value using a random forest algorithm, based on the parameters and corresponding time windows presented in Table 1:

| Electro- | | | | |
|---|---|---|---|---|
| dermal | Cardiac | Eye-tracking | Behavioral | Personality |
| 0-60 s | 0-60 s | 0-30 s | 0-60 s | |
| N_scrs | SDNN | nbFix | SDLP | Openness |
| Amp_moy | | durationFixTot | SDWA | |
| Et_Amp_scrs | | durationFixMean | | |
| Lat_Moy_scrs | | durationFixSD | | |
| ET_lat_scrs | | | | |

When validated on the 10 final test subjects, the method had a true positive rate of 0.744, a false positive rate of 0.262, a precision of 0.703, recall of 0.744, an F-measure of 0.744, a Matthews correlation coefficient of 0.485 and areas of 0.826 and 0.845 under, respectively, the receiver operating characteristic and precision recall curves.

In the second example, the data processing device 1 determined the discomfort level index value using a J48 algorithm, based on the parameters and corresponding time windows presented in Table 2:

| Electro- | | | | |
|---|---|---|---|---|
| dermal | Cardiac | Eye-tracking | Behavioral | Personality |
| 0-15 s | 0-60 s | 0-60 s | 0-120 s | |
| ET_lat_scrs | SDNN | durationFixTot | SDWA | Openness |
| Amp_moy | | | | Agreeability |
| | | | | Extraversion |
| | | | | Neuroticism |

When validated on the 10 final test subjects, the method had then a true positive rate of 0.698, a false positive rate of 0.296, a precision of 0.704, recall of 0.698, an F-measure of 0.698, a Matthews correlation coefficient of 0.402 and areas of 0.739 and 0.704 under, respectively, the receiver operating characteristic and precision recall curves.

In the third example, the data processing device 1 determined the discomfort level index value using a naïve Bayesian algorithm, based on the parameters and corresponding time windows presented in Table 3:

| Electro- | | | | |
|---|---|---|---|---|
| dermal | Cardiac | Eye-tracking | Behavioral | Personality |
| 0-15 s | 0-60 s | 0-30 s | 0-60 s | |
| N_scrs | SDNN | durationFixTot | SDLP | Openness |
| | | | SRR | Neuroticism |

When validated on the 10 final test subjects, the method had then a true positive rate of 0.744, a false positive rate of 0.262, a precision of 0.747, recall of 0.744, an F-measure of 0.742, a Matthews correlation coefficient of 0.486 and areas of 0.743 and 0.757 under, respectively, the receiver operating characteristic and precision recall curves.

Those skilled in the art will recognize that the present disclosure may be manifested in a variety of forms other than the specific embodiments described and contemplated herein. Accordingly, departure in form and detail may be made without departing from the scope of the present disclosure as described in the appended claims.

What is claimed is:

1. A data processing device configured to be connected to one or more physiological sensors for receiving physiological data of a user, to one or more behavioral sensors for receiving behavioral data of the user and to a vehicle context detection unit for receiving vehicle context information, and to determine a discomfort level index value based on a personality profile of the user together with at least the vehicle context information, the physiological data and the behavioral data received within corresponding time windows.

2. The data processing device according to claim 1, wherein the data processing device is configured to be also connected to an emotional state detection unit for receiving emotional state information concerning the user, and to determine the discomfort level index value based also on the emotional state information concerning the user.

3. The data processing device according to claim 2, wherein the emotional state detection unit is configured to detect emotional cues in images and/or sound received from a camera and/or a microphone connected to the emotional state detection unit.

4. The data processing device according to claim 1, wherein the personality profile comprises an index value associated to each of one or more personality traits.

5. The data processing device according to claim 4, wherein the one or more personality traits include openness, agreeableness, extraversion, neuroticism, conscientiousness, and/or trait anxiety.

6. The data processing device according to claim 1, wherein the data processing device is configured to apply a learning classification algorithm for determining the discomfort level index value.

7. The data processing device according to claim 6, wherein the learning classification algorithm is any one of random forest, J48, naïve Bayesian, deep neural network, or recurrent neural network algorithms.

8. The data processing device according to claim 1, wherein the one or more physiological sensors include one or more electro-dermal sensors.

9. The data processing device according to claim 8, wherein the data processing device is configured to receive, as the physiological data from the one or more electro-dermal sensors, occurrence, amplitude and/or latency of phasic skin conductivity responses.

10. The data processing device according to claim 1, wherein the one or more physiological sensors include one or more cardiac sensors.

11. The data processing device according to claim 10, wherein the data processing device is configured to receive RR intervals as the physiological data from the one or more cardiac sensors.

12. The data processing device according to claim 1, wherein the one or more physiological sensors include one or more eye tracking sensors.

13. The data processing device according to claim 12, wherein the data processing device is configured to receive occurrence and/or duration of eye fixations as the physiological data from the one or more eye tracking sensors.

14. The data processing device according to claim 1, wherein the data processing device is configured to output a warning and/or trigger user assistance if the discomfort level index value exceeds a predetermined threshold.

15. A human-machine interface system comprising the data processing device according to claim 1, as well as the one or more physiological sensors, the one or more behavioral sensors, and the vehicle context detection unit, each connected to the data processing device.

16. A vehicle comprising the human-machine interface system according to claim 15

17. A computer-implemented method for evaluating user discomfort, comprising the steps of:
receiving physiological data of a user from one or more physiological sensors;
receiving behavioral data of the user from one or more behavioral sensors;
receiving vehicle context information from a vehicle context detection unit; and
determining a discomfort level index value based on a personality profile of the user, together with at least the physiological data, the behavioral data and the vehicle context information received within corresponding time windows.

18. A computer-readable medium comprising instructions which, when executed by a computer, cause the computer to carry out the computer-implemented method according to claim 17.
